(19) United States
(12) Patent Application Publication
(10) Pub. No.: US 2021/0212611 A1
(43) Pub. Date: Jul. 15, 2021

(54) OXIMETRY SYSTEM SELF-TEST

(71) Applicant: Nonin Medical, Inc, Plymouth, MN (US)

(72) Inventors: Gregory J. Rausch, Minnetonka, MN (US); Matthew Prior, Plymouth, MN (US); Charles U. Smith, Plymouth, MN (US)

(21) Appl. No.: 17/059,972

(22) PCT Filed: May 31, 2019
(86) PCT No.: PCT/US19/34884

§ 371 Date: Nov. 30, 2020

Related U.S. Application Data
(60) Provisional application No. 62/679,253, filed on Jun. 1, 2018.

(51) Int. Cl. A61B 5/1455; A61B 5/1495

(52) U.S. Cl. CPC A61B 5/14551; A61B 2560/0233; A61B 5/1495

(57) ABSTRACT

An oximeter can include a sensor and a processor. The processor can access signal information corresponding to a test signal. The signal information can correspond to a characteristic. The characteristic can be determined using at least one of an AC component and a DC component. The processor can compare the characteristic and a reference value. The processor can adjust the signal, based on the comparison, to set a relationship between the characteristic and the reference value.

### CLAIM OF PRIORITY

This application claims the benefit of priority to U.S. Patent Application Ser. No. 62/679,253, filed on Jun. 1, 2018, which is incorporated by reference herein in its entirety.

### BACKGROUND

A pulse oximeter typically includes a sensor having a photodetector and an emitter. The emitter (such as a light-emitting diode, LED), in combination with the photodetector, provides a measurement of oxygenation in a patient. To ensure measurement accuracy, the pulse oximeter can be tested or calibrated. Current techniques for testing the pulse oximeter are inadequate.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document. Such embodiments are demonstrative and not intended to be exhaustive or exclusive embodiments of the present apparatuses, systems, or methods.

FIG. 1 illustrates an example of portions of an oximeter, according to one example.

FIG. 2 illustrates selected aspects of a patient signal, according to one example.

FIG. 3 illustrates an example of a method associated with an oximeter, according to one example.

FIGS. 4A, 4B and 4C illustrate graphical examples of a signal, according to one example.

FIG. 5 illustrates an example of a method associated with an oximeter, according to one example.

FIG. 6 illustrates an example of a method associated with an oximeter, according to one example.

FIG. 7 illustrates a schematic of an oximeter, according to one example.

FIG. 8 illustrates an example of tabulated data associated with an oximeter.

FIG. 9 illustrates a system, according to one example.

FIG. 10 illustrates a calibration unit, according to one example.

FIG. 11 illustrates a system, according to one example.

### DETAILED DESCRIPTION

An oximeter can be configured to perform a self-test using a test signal representative of a patient signal. The oximeter can generate the signal and modulate the emitted light (from an emitter). The self-test can be a functional self-test or a calibration self-test. In an example, the self-test can include a closed-loop (feedback) test of the oximeter functionality.

FIG. 1 illustrates an example of portions of oximeter 100. In the example shown, oximeter 100 includes sensor 102, driver 110, amplifier 112, processor 118, memory 126, interface 130, and clock 128. Oximeter 100 can be configured to determine pulse oximetry, regional oximetry (sometimes referred to as tissue oximetry) or other parameter suited for measurement with near infrared stimulation and optical detection.

Sensor 102 includes emitter 104, optical attenuator 106, and detector 108. Emitter 104 can include one emitter or a plurality of emitters, any of which can include a light emitting diode. In one example, attenuator 106 provides a fixed attenuation to optical energy. In one example, attenuator 106 can include an optical element having a selectable attenuation wherein a parameter associated with the attenuation is determined by an electrical terminal coupled to the attenuator. The parameter can be a magnitude, phase, frequency, or timing (such as a duty cycle) of attenuation. Detector 108 can include a photodetector or other optical detector.

Emitter 104 is coupled to driver 110 and driver 110 is coupled to processor 118. Processor 118 can provide a signal to driver 110 which controls the intensity of emitted light from emitter 104.

Attenuator 106 is coupled to processor 118. In one example, processor 118 can provide a signal to modulate or control light emitted from emitter 104. For example, processor 118 can adjust a drive current supplied to emitter 104 or adjust a duty cycle of electric current supplied to emitter 104. The light emitted from 104 can be controlled to ensure that a photodiode current of a corresponding detector 108 can be varied across the expected received signal levels. The signal levels can vary based on factors such as size and absorption across the human finger population, for example.

Processor 118 is coupled to amplifier 112 and amplifier 112 is coupled to detector 108. Amplifier 112 has a selectable gain 114 and a selectable reference 116. Processor 118 can provide a signal for the selection of amplifier gain and can provide a signal for selection of a reference value (such as current, resistance voltage). An output signal from detector 108 is amplified at a particular gain determined by gain 114 and can be compared with a reference level determined by reference 116.

Processor 118 is coupled to interface 130. Interface 130 can include a user interface having an input device (such as a user-operable switch, keyboard, or mouse) and an output device (such as a visual display or a port for connection to a network, communication channel, a printer port, wireless transceiver) or other input or output device.

Processor 118 is coupled to memory 126 Memory 126 can provide storage for instructions for execution by processor 118 or storage for data.

Clock 128 is coupled to processor 118 and can provide a timing signal to control operation of processor 118.

Processor 118 is coupled to amplifier 112. In an example, processor 118 can modulate an amplifier gain 114 or an amplifier reference 116, such as to modulate the signal (e.g., a simulated patient signal or a test signal). In an example, processor 118 can be configured to modulate the drive signal provided to emitter 104 by driver 110, such as to modulate the signal. The oximeter 100 can include other elements, such as other sensors, a filter, an analog-to-digital converter (ADC) or a digital-to-analog converter (DAC).

In one example, processor 118 can be configured to control light transmissivity of attenuator 106. Attenuator 106 can be modulated to simulate attenuation of light corresponding to tissue, such as a human finger, positioned in a light path between emitter 104 and detector 108.

In an example, emitter 104 is configured to emit light. Detector 108 can include a photodetector. Emitter 104 is driven at a specified current level over time, such as by using the emitter driver 110.

In an example, sensor 102 includes one detector 108 and multiple emitters (e.g., more than one emitter, eight emitters, or more than eight emitters). Each of the multiple emitters can be driven at a different current level overtime. The multiple emitters are time multiplexed and the emitted light is received by the detector 108.

In an example, sensor 102 includes multiple detectors (e.g., more than one detector, eight detectors, or more than eight detectors) and multiple emitters 104. Each of the multiple detectors can provide an output signal, sometimes referred to as a wavelength signal. The multiple emitters can be configured to illuminate in sequence (e.g., a first, a second, a third, and so on). The multiple emitters can be driven at a repetition rate (e.g., sampling rate) that is configured for measuring the pulsatile component of the patient signal. In one example, a 75 Hz emitter drive repetition rate is used. A 75 Hz emitter drive repetition rate provides a particular resolution in a digitized measurement. A 75 Hz emitter drive repetition rate allows for time multiplexing of the multiple wavelength signals provided by the multiple detectors. The emitter drive repetition rate can be less than 75 Hz or greater than 75 Hz. Some examples of factors that can affect the output signal provided by the detector include the emitter 104 drive current, efficiency of the emitter 104, tissue absorption of light, or scattering. Some of these factors, such as the drive current for example, can be controlled to provide a desired output. In an example, the multiple emitters are illuminated in a non-uniform sequence (e.g., a first, a second, a third, the first, the third), or another sequence specified by a user.

In an example, oximeter 100 can be used on a patient, such as on a digit (finger, toe) of the patient. During operation of oximeter 100 on the patient, an output of the sensor 102 includes an ambient light signal component and a patient signal component. The patient signal component can include an alternating current (AC) component and a direct current (DC) component. The AC component can be representative of the patient's pulse (e.g., pulsatile arterial blood absorption). The DC component can be representative of the non-pulsatile absorption (e.g., tissue or venous blood absorption).

In an example, the optical attenuator 106, disposed between the emitter 104 and the detector 108, can be modulated to increase or decrease the power level of the test signal. Modulating optical attenuator 106 can include changing a light transmissivity property. In an example, an analog-to-digital converter can be configured to convert a test signal from a measured continuous physical quantity (e.g., such as measured in voltage), as measured by detector 108, to a digital value. In an example, a digital-to-analog converter can convert digital data from processor 118 or driver 110 into an analog signal (e.g., such as a current or voltage). The test signal provided by the detector 108 can be conveyed to the processor 118. The processor 118 can access executable instructions and data stored in memory 126. A portable or metered power service can provide electric energy to processor 118 and other elements of the oximeter 100.

FIG. 2 illustrates a pictorial example of patient signal 208, such as during operation of oximeter 100 with a patient. The horizontal axis can represent time (such as measured in seconds). The vertical axis can represent a sensor output signal. The sensor output signal can correspond to light attenuation or light absorption. Detector 108 can be configured to provide an output signal corresponding to reflected light or transmitted light. Light detected by detector 108 is affected by noise, scattering, reflection, and other factors. In one example, detector 108 provides a signal 210 corresponding to light that was not attenuated by patient tissue and emitted by emitter 104. The patient signal 208 can represent the light that has been absorbed by the patient's finger, for example, and has an AC component 202 and a DC component 204. The DC component 204 can represent the light absorbed by the tissue and venous blood. The AC component 202 can represent the light absorbed by pulsatile arterial blood. In this example, the ambient light signal 206 has a 1 nanoampere (nA) to 100 microampere (μA) current range. The patient signal component 208 includes the AC component 202 and the DC component 204. In this example, the patient signal corresponds to a 1 μA to 50 μA current range. The patient signal 208 can have a percent modulation (also referred to as % modulation or modulation percentage), such as 0.001% to 10%.

FIG. 3 illustrates an example of method 300 for using oximeter 100. At 302, signal information is accessed, such as by using the processor 118. In an example, the signal information can be stored data, such as stored using memory 126. In an example, the signal information can be accessed in real-time or near real-time. The signal information can include information about a characteristic of the test signal. The test signal is generated using sensor 102. The characteristic can be associated with an amplitude, a phase, a frequency, a power level, a wavelength, a pulse width, or another signal characteristic. The characteristic can be associated with the percent modulation. The AC and DC components can each have a respective characteristic.

The percent modulation can be determined for the signal. The percent modulation can be determined for each of the multiple wavelength signals. In an example, the percent modulation is determined by a ratio of the AC component and the DC component, such as can be determined using a current, voltage, absorbance, or another measurement of the AC and DC components. The characteristic can be associated with other relationships, such as a function or a mathematical equation (e.g., an average or a weighted average), as to the AC and DC components.

At 304, the characteristic of the test signal and a reference value are compared, such as by using processor 118. The reference value can be associated with a physiological measurement of the patient. In an example, the reference value can be associated with carboxyhemoglobin (COHb) level, methemoglobin (MetHb) level, regional oxygen saturation level (rSO₂), or peripheral capillary oxygen saturation (SpO₂) level (also referred to as oxygen saturation, patient oxygenation, or blood oxygenation). Other patient physiological measurements can be associated with the reference value. In one example, the reference value can be associated with known values of SpO₂, COHb, or MetHb, such as obtained during known conditions of the patient. In an example, the reference value can be obtained using information from a clinical trial. In yet another example, the reference value can be specified by the user, such as a health care provider. In an example, the reference value is associated with a percent modulation of the signal under known conditions. The characteristic, such as from the test signal, is compared to the reference value. The reference value can be associated with multiple wavelength signals. The reference value can be associated with a function or a mathematical equation, such as measured during known conditions, such that the characteristic and the reference value can be compared. The comparison can result in a difference, or a quantitative relationship between the characteristic and the reference value. In an example, the result of the comparison can be a change between the characteristic and the reference value overtime. The result of the comparison can be associated with the signal information.

At 306, the test signal is adjusted based on the comparison, such as by using processor 118. Processor 118 can use the signal information, including the comparison between the characteristic of the signal and the reference value. Processor 118 can adjust elements of the oximeter 100, such as emitter driver 110 or amplifier 112, to adjust the signal. In an example, the oximeter 100 is calibrated by adjusting the test signal, based on using the reference characteristic. In an example, the test signal is a simulated patient signal that is adjusted to desired parameter levels, such as for use as a functional self-test of the oximeter 100.

In one example a compensation value is generated and provided to a user. The user can make an adjustment in a measured value based on the compensation value and thus, determine a compensated measure of oxygenation.

Processor 118 can be configured to modulate (e.g., vary or adjust) the emitter drive current, or the output of the amplifier 112, such as to modulate the test signal generated by the sensor 102. Processor 118 can be configured to modulate other components of the oximeter 100. Oximeter 100 can be configured to modulate the test signal with or without an external device, such as a simulator device.

In an example, during operation of the oximeter 100 on a patient, the percent modulation measurements of the various wavelength signals are used to calculate a value for SpO₂, COHb, and MetHb. In an example, during the self-test of the oximeter 100, by adjusting the test signal based on the comparison, oximeter functional testing can be improved. The AC and DC components can be unique for each of the multiple wavelength signal. The adjusted signal can be representative of a patient signal that has specified levels of physiological measurements, such as SpO₂, COHb, MetHb, pulse rate, pulse amplitude, DC absorption, or other physiological measurements.

In an example, each of the multiple wavelength signals has a respective characteristic. In an illustrative example with eight wavelength signals, there can be eight respective characteristics that correspond to each of the eight wavelength signals. For each reference value that oximeter 100 is calibrated with, the set of characteristics (e.g., percent modulations) associated with the multiple wavelength signals are maintained with reference to one another.

In an example, the percent modulations of the multiple wavelength signals are maintained relative to one another. The DC component is modulated to the desired amplitude, such as an amplitude representative of a normal patient signal or associated with a reference value. In an example of a device with eight emitters (e.g., eight wavelength signals), by modulating the AC component, the percent modulations of the multiple wavelength signals can be maintained relative to one another. A pulse amplitude of each wavelength signal is specified. The frequency value of one of the wavelength signals is set to an infrared (“IR”) value or range. The percent modulation of the IR wavelength signal is set equal to the desired pulse amplitude for the simulated signal. The percent modulation for the other seven wavelength signals, in this example, is determined relative to the percent modulation of the IR wavelength signal. In an example, the percent modulation for the other seven wavelength signals (e.g., wavelength signals 1-7) is calculated using a ratio between wavelength signals 1-7 percent modulation and the IR wavelength signal percent modulation. For each of the eight wavelength signals, the respective AC component amplitude is calculated using signal information, such as the information about the desired amplitude of the DC component, or the information about the respective percent modulations. A morphology of the AC component is determined. The wave shape of the AC component can be the same or similar for each of the wavelength signals. In an example, the duration of the wave shape of the AC component can be associated with the pulse rate. The amplitude and wave shape of the AC component for each of the wavelength signals is digitized using signal information, such as the signal sampling rate of the oximeter 100.

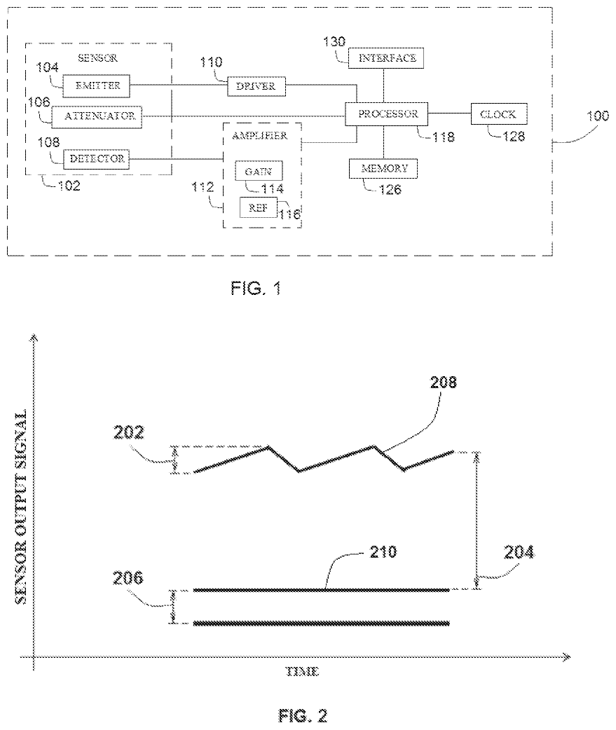

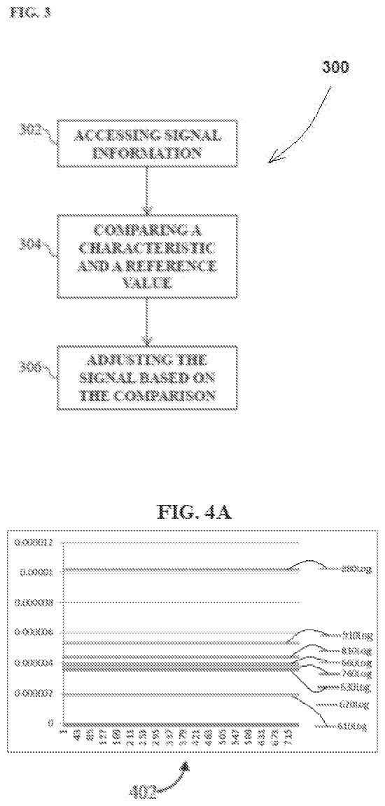

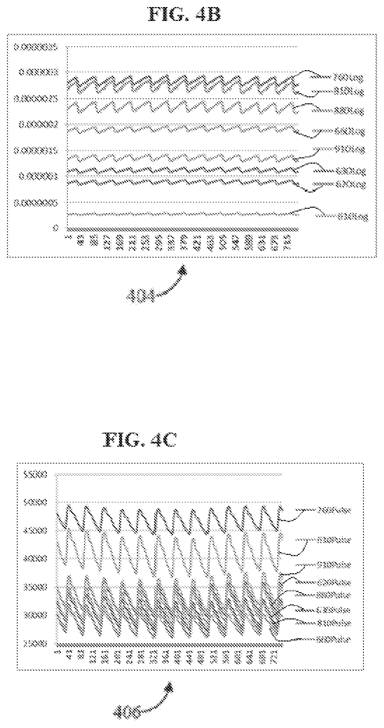

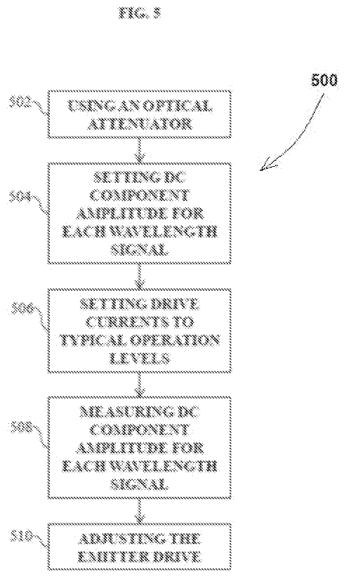

FIG. 4A illustrates a graphical example of components of the signal. In graph 402, the DC component for each of the multiple wavelength signals are illustrated over time.

FIG. 4B illustrates a graphical example of components of a signal. In graph 404, the DC and AC components for each of the multiple wavelength signals are illustrated over time. AC component modulation of the DC component is illustrated.

FIG. 4C illustrates a graphical example of components of a signal. In graph 406, the DC component and an amplified AC component for each of the multiple wavelength signals are illustrated overtime. Amplified AC component modulation of the DC component is illustrated.

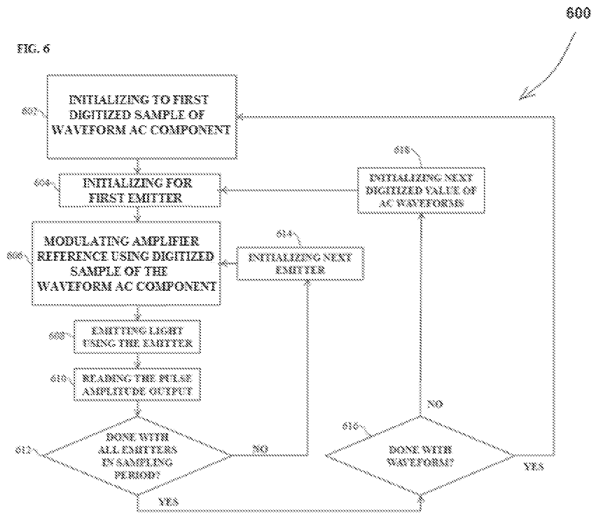

FIG. 5 illustrates an example of method 500 for using an oximeter. At 502, an optical attenuator, such as the optical attenuator 106, is used. In an example, the optical attenuator 106 is used to attenuate light output that is received by the detector 108. Use of the optical attenuator 106 can result in a DC component of the signal that simulates (e.g., approximates) a DC component of a typical patient signal. In an example, the processor 118 is configured to adjust the emitter drive 110, such as to modulate the light output that is received by the detector 108. This provides a closed loop test of the oximeter 100, as the light output of the emitter 104 reaches the detector 108.

At 504, a DC component amplitude for each of the multiple wavelength signals is set. At 506, the emitter drive current is set to typical operation levels (e.g., levels used during operation of the oximeter on the patient), such as by using the processor 118. The processor 118 is configured to set the emitter drive 110 to the typical operation level. In an example, the processor 118 is configured to set a respective amplitude for each of the multiple wavelength signals, such as by setting the drive current for multiple emitters.

At 508, the DC component for each wavelength signal is measured, such as by using the processor 118. At 510, the emitter drive 110 is adjusted, such as by using the processor 118. By adjusting the emitter drive 110, the desired DC component amplitude level is obtained. In an example, the respective drive current for each of the multiple wavelength signals is adjusted, such as by using respective emitter drivers and respective emitters. In this way, the desired respective DC component amplitude levels for each of the multiple wavelength signals are obtained.

FIG. 6 illustrates an example of method 600 for using oximeter 100. At 602, method 600 includes initializing to the first digitized sample of the waveform AC component. Prior to 602, the AC component of the signal has been digitized at the signal sampling rate (e.g., 75 Hz emitter drive repetition rate). In an example, the AC component is provided, such as by using amplifier 112.

At 604, method 600 includes initializing for the first emitter. In an example, the respective emitters for each of the multiple wavelength signals (e.g., signals 1-8) are illuminated. At 606, amplifier reference 116 is modulated, such as by using processor 118, using a subsequent (e.g., next) digitized sample of the waveform AC component. At 606, for example, a digital-to-analog converter is used to set the amplifier reference 116. In an example, the amplifier reference 116 is modulated at a desired rate, amplitude, or shape, such as to generate a signal having the desired AC components. At 608, light is emitted using the emitter 104 in sequence. At 610, a pulse amplitude output is read (e.g., determined or measured). In an example, the modulation of the amplifier reference 116 continues for each respective emitter for each of the multiple wavelength signals.

At 612, it can be determined whether the modulation of the amplifier reference 116 has been done for each respective emitter for each of the multiple wavelength signals for a particular sampling period (e.g., 75 Hz sampling period). If the answer at 612 is no, then at 614, the next respective emitter is initialized. The amplifier reference 116 is modulated for each respective emitter. After initializing the next emitter at 614, the method continues to element 606.

If the answer at 612 is yes, then at 616, it can be determined whether the waveform is done, such as by using the processor 118. If the answer at 616 is no (e.g., a full waveform used for the self-test is not yet complete), then at 618, the next digitized value of the respective AC components for each of the multiple wavelength signals is initialized.

If the answer at 616 is yes, then the method continues to element 602. In this example of using the oximeter 100, such as shown in FIG. 6, the method can be performed iteratively, such as to provide continuous operation (e.g., “closed loop” operation).

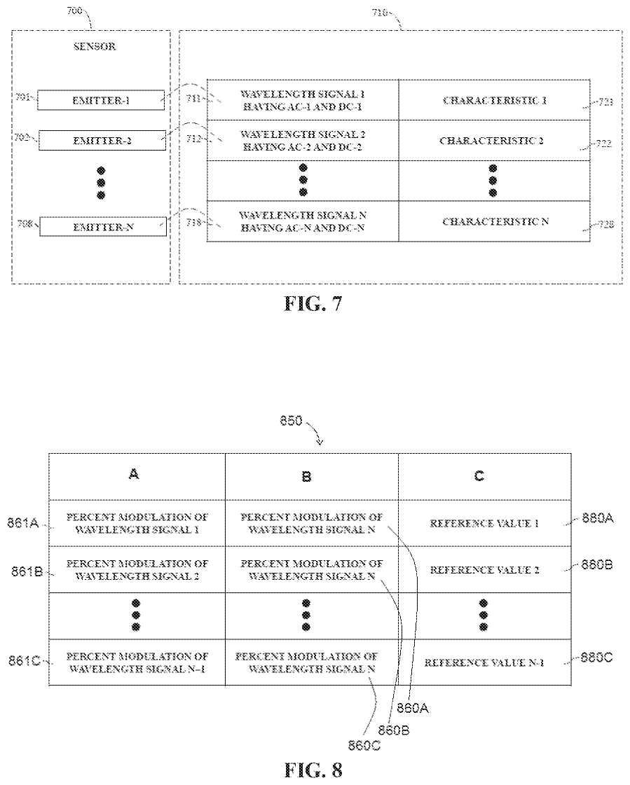

FIG. 7 illustrates an example of using an oximeter. Element 700 illustrates portions of a sensor, such as the sensor 102. Element 700 includes multiple emitters (e.g., emitter-1 701, emitter-2 702, through emitter-N 708). As described elsewhere in this document, N can equal eight or another specified number of emitters. Each of the multiple emitters can be configured to emit light having specified parameters (e.g., wavelength or frequency).

Element 710 illustrates conceptually, by way of example, signal information of a test signal having multiple wavelength signals. The multiple wavelength signals can correspond to respective emitters. In this manner, element 711 of wavelength signal 1 having AC component 1 and DC component 1 (e.g., AC-1 and DC-1) can correspond to emitter-1701, element 712 of wavelength signal 2 having AC component 2 and DC component 2 (e.g., AC-2 and DC-2) can correspond to emitter-2 702, and element 718 of wavelength signal N having AC component N and DC component N (e.g., AC-N and DC-N) can correspond to emitter-N 708. Each of the multiple wavelength signals can correspond to a respective characteristic, such as shown at characteristic-1 721, characteristic-2 722, and characteristic-N 728 in FIG. 7. In one example, each respective characteristic corresponds to a percent modulation, such as determined using the AC component and DC component for each wavelength signal.

In one example, each respective characteristic corresponds to a ratio between the percent modulation of that particular wavelength signal and a different percent modulation of a different wavelength signal.

FIG. 8 illustrates an example of tabulated data associated with using an oximeter. In an example, table 850 conceptually illustrates how signal information is used in the self-test of the oximeter. Column A includes the percent modulation for each of the multiple wavelength signals from 1 to N−1. Column B includes the percent modulation for wavelength signal N. In an example, wavelength signal N is set to an IR value or range. Column C includes reference values that correspond to the respective wavelengths for each of the multiple wavelength signals.

As described elsewhere, for example, the reference values of Column C can be associated with known values of SpO₂, total hemoglobin (tHb), respiration rate, COHb, or MetHb, such as obtained during known conditions of the patient. In an example, the reference values can be obtained using information from a clinical trial. A set of reference values (e.g., 880A, 880B, and 880C) can be associated with a particular physiological measurement, such as COHb for example. The reference value can be a ratio between two percent modulations of specified wavelength signals (e.g., a ratio between Column A and Column B). In this manner, for each of the specified wavelength values for multiple wavelength signals, there can be a specified reference value that can be used to modulate the test signal to perform the self-test.

In an example, during the self-test of the oximeter, processor 118 can access information (such as the information of Column A and Column B of FIG. 8) from memory 126. For example, processor 118 can determine the ratio between the percent modulation of each of the multiple wavelength signals (e.g., elements 861A, 861B, and 861C) and the percent modulation of an IR wavelength signal (e.g., percent modulation of wavelength signal N 860A, 860B, and 860C). The determined ratio (e.g., “characteristic”) can be compared to the reference value. The test signal can be adjusted, based on the comparison, to set a predetermined relationship between the characteristic and the reference value.

In an example, optical attenuator 106 can be used to modulate the test signal (e.g., the DC component of the test signal). In an example, the emitter drive 110 can be used to modulate the DC component. Other elements can be used to modulate the DC component. The emitter drive 110 is modulated to reduce the emitter drive current, such as to cause a DC component to be lowered into a range that can be found in the typical patient signal.

In an example, the amplifier gain 114 can be used to modulate the AC component. The amplifier gain 114 can be modulated at a desired rate, amplitude, and wave shape, such as to cause the AC component to be modulated to be representative of a typical patient signal. In an example, the amplifier reference 116 (e.g., an amplifier reference input) can be used to modulate the AC component. In an example, the emitter drive 110 can be can be modulated at a desired rate, amplitude, and wave shape, such as to cause the AC component to be modulated to be representative of a typical patient signal.

In an example, the oximeter 100 measures the sensor output signal during the period when light is emitted from the sensor emitter and subtracts the ambient signal component to obtain the patient signal, such as during operation of the oximeter 100 on a patient.

In an example, the present subject matter is useful for improving oximeter functionality or methods of using an oximeter, such as providing an oximeter self-test. The present subject matter can be used for other types of sensor systems (e.g., a regional oximetry device, such as configured for sensing regional oxygenation or “RSO₂”). In an example, the power 120 can be a battery configured inside the oximeter 100 (e.g., as shown in FIG. 1), or the power 120 can come from a power outlet.

In an example, the present subject matter can provide a functionality test for an oximeter that measures COHb. In an example, the present subject matter can include a remote device. The remote device can include a wireless transceiver configured to wirelessly communicate with a wireless transceiver of a controller. The controller can transmit to the remote device, and the remote device can transmit to the controller. In an example, the present subject matter can include a monitor, such as to provide audio or visual indications to a user, such as a health care provider. In an example, the oximeter 100 can provide information to an external device, such as by using the interface 130 (e.g., such as to provide information to a treatment device). In various examples, interface 130 can include a network connection or a wireless telemetry module.

In an example, the optical attenuator 106 is configured to provide an absorption coefficient that is spectrally flat, or has a known profile, in a particular wavelength range or value. In an example, the processor can be configured to modulate the optical attenuator 106 in order to modulate the test signal uniformly across multiple wavelengths.

In one example, multiple wavelength signals may be time multiplexed and emitted light can be received by a single detector. In this manner, a single detector can be associated with multiple signals.

FIG. 9 illustrates a block diagram of system 900A. System 900A includes monitor 910A, calibration unit 920A, and sensor 930A. In the example shown, system 900A includes remote device 940.

Monitor 910A, calibration unit 920A, and sensor 930A can include a combination of elements shown in FIG. 1. For example, monitor 910A can include interface 130, processor 118, memory 126, clock 128 and driver 110, and in a similar manner, calibration unit 920A can include a processor, a memory, and selected elements such as those shown in FIG. 1.

Sensor 930A is shown to include an emitter (E), a detector (D) and an intermediary attenuator (A). The attenuator is positioned in a light path between the emitter and the detector. In various examples, the attenuator can include a foam-filled cavity, a liquid filled cavity, or a composite material selected to introduce attenuation to a near infrared wavelength optical signal.

Calibration unit 920A can communicate with monitor 910A via wired connector 924 or via wireless telemetry as represented by antenna 926.

In the example shown, calibration unit 920A provides an interface between monitor 910A and sensor 930A. In one example, calibration unit 920A is configured to communicate wirelessly with remote device 940. Remote device 940 can include a multi-parameter system, a cellular telephone, a tablet computer, a desktop computer, or other device.

In one example, calibration unit 920A is configured to determine identity of a manufacturer, model, or type of monitor 910A or determine identity of a manufacturer, model, or type of sensor 930A. The identity information can be encoded in a component value, a measured parameter, a signal level, or encoded in a memory of the corresponding unit. Calibration information, as determined by calibration unit 920A, can be associated with identity of the monitor 910A or of the sensor 930A and stored in an internal memory for later use. For example, trending statistics can be determined over a lifetime of a unit and detect LED power level fade or sensor deterioration.

Calibration unit 920A can be configured to simulate a particular parameter associated with a skin tone, a perfusion level, an artifact or noise level (such as motion, jogging, a tremor, sleep, or other activity).

In one example, calibration unit 920A can be configured to modulate sensor 930A or modulate data provided to monitor 910A in order to generate calibrated values for a selected measure of oximetry or other parameter detected by sensor 930A. For example, calibration unit 920A can modulate an attenuator, an emitter drive current, or a detector output signal to align the sensor-supplied signal levels with a calibrated standard. In another example, calibration unit 920A can provide a suitable signal to monitor 910A in order to provide a true measure of oximetry or other parameter, based on a calibration value and based on a sensed signal provided by sensor 930A.

In one example, calibration unit 920A includes a visual display to provide a visible indication to an operator. The visible indication can be manually applied to the measured data as displayed by monitor 910A in order to compensate for a noise or other artifacts.

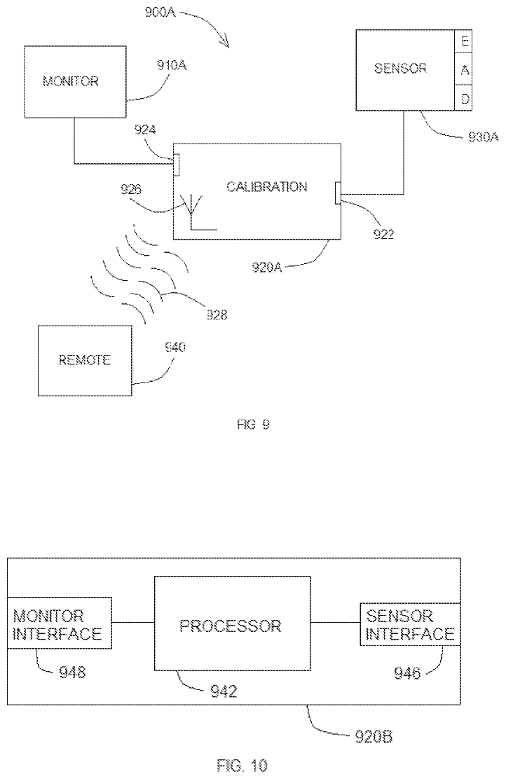

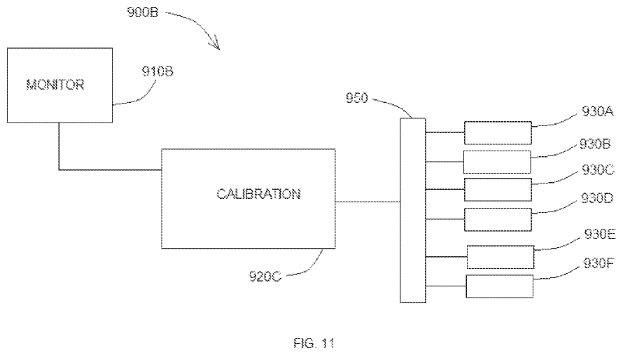

FIG. 10 illustrates calibration unit 920B, according to one example. Calibration unit 920B includes monitor interface 948 and sensor interface 946. Monitor interface 948 can include connector 924 and antenna 926 and sensor interface 946 can include connector 922, as described relative to system 900A.

Processor 942 can include a memory and a user interface. Processor 942 can be configured to perform a variety of methods including determining identity of a sensor or a monitor. In one example, processor 942 can be configured to receive user input as to skin tone, or activity (either manually entered by a user or provided by an accelerometer) and calculate calibration information suitable for determining a compensation factor dishemoglobin or other measured parameter.

FIG. 11 illustrates a block diagram of system 900B. System 900B includes monitor 910B, calibration unit 920C, hub 950, and sensors 930A-930F. Hub 950 provides a common bus and interface with which monitor 910B (and calibration unit 920C) can communicate with sensors 930A-930F individually or in any combination. According to one example, calibration unit 920C can be configured to conduct a self-test on each of sensors 930A, 930B, 930C, 930D, 930E, and 930F in serial or parallel manner. The example illustrated depicts six sensors however, any number of sensors can be used.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided.

Each of these non-limiting examples can stand on its own, or can be combined in various permutations or combination with one or more of the other examples.

Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, an apparatus, system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A method for using an oximeter, the method comprising:
accessing signal information corresponding to a test signal, the test signal being representative of a patient signal, and the test signal including at least one of an AC component and a DC component, wherein the signal information corresponds to a characteristic of the test signal, and wherein the characteristic is determined using at least one of the AC component and the DC component;
comparing the characteristic and a reference value; and
adjusting the test signal, based on the comparing, to set a relationship between the characteristic and the reference value.

2. The method of claim 1, wherein accessing the signal information includes accessing a percent modulation of the test signal, wherein the percent modulation corresponds to a ratio of the AC component and the DC component.

3. The method of claim 1, wherein adjusting the test signal includes modulating transmissivity of light using an optical attenuator.

4. The method of claim 1, wherein adjusting the test signal includes modulating an amplifier reference.

5. The method of claim 1, wherein adjusting the test signal includes modulating an emitter drive.

6. The method of claim 1, wherein adjusting the test signal includes modulating an amplifier gain.

7. The method of claim 1, wherein the test signal is comprised of multiple wavelength signals, each of the multiple wavelength signals associated with a respective detector, each of the multiple wavelength signals having a respective AC component and a respective DC component, and each of the multiple wavelength signals having a respective characteristic determined using the respective AC component and the respective DC component.

8. The method of claim 7, wherein adjusting the test signal includes adjusting each of the multiple wavelength signals to maintain a relationship among the respective characteristics of each of the multiple wavelength signals.

9. The method of claim 1, comprising iterating the comparing multiple times, and adjusting the test signal to set the relationship between the characteristic and the reference value in a feedback loop.

10. The method of claim 1, wherein the reference value corresponds to at least one of a carboxyhemoglobin value, a methemoglobin value, or an oxygenation value.

11. An oximeter comprising:
a sensor configured to generate a test signal, the test signal representative of a patient signal, and the test signal including at least one of an AC component and a DC component;
a processor, coupled to the sensor, the processor configured to:
access signal information corresponding to the test signal, the signal information corresponding to a characteristic, and wherein the characteristic is determined using at least one of the AC component and the DC component;
compare the characteristic and a reference value; and
adjust the test signal, based on the comparison, to set a relationship between the characteristic and the reference value.

12. The oximeter of claim 11, comprising an optical attenuator coupled to the sensor, the sensor including an emitter, and the optical attenuator configured to modulate transmissivity of light emitted from the emitter.

13. The oximeter of claim 11, comprising an emitter drive coupled to the sensor, wherein the processor is configured to modulate the emitter drive to adjust at least one of the AC component and the DC component.

14. The oximeter of claim 11, comprising an amplifier coupled to the sensor, wherein the processor is configured to modulate a reference of the amplifier to adjust the AC component.

15. The oximeter of claim 11, comprising an amplifier coupled to the sensor, wherein the processor is configured to modulate a gain of the amplifier to adjust the AC component.

16. The oximeter of claim 11, wherein the sensor includes at least one detector, the sensor configured to provide at least one wavelength signal using the at least one detector, wherein each at least one wavelength signal includes a respective AC component, a respective DC component, and a respective characteristic.

17. The oximeter of claim 11, wherein the processor is configured to access signal information including a percent modulation of the test signal, wherein the percent modulation corresponds to a ratio of the AC component and the DC component.

18. A system for using an oximeter, the system comprising:
a processor configured to:
access signal information corresponding to a test signal, the test signal representative of a patient signal, and the test signal including at least one of an AC component and a DC component, wherein the signal information corresponds to a characteristic, and wherein the characteristic is determined using at least one of the AC component and the DC component;
compare the characteristic and a reference value; and
adjust the test signal, based on the comparison of the characteristic and the reference value, to set a relationship between the characteristic and the reference value.

19. The system of claim 18, wherein the processor is configured to access the characteristic, wherein the characteristic corresponds to a percent modulation of the signal, and wherein the percent modulation corresponds to a ratio of the AC component and the DC component;
wherein the test signal is comprised of multiple wavelength signals, wherein each of the multiple wavelength signals includes a respective AC component, a respective DC component, and a respective characteristic determined using the respective AC component and the respective DC component; and
wherein the processor is configured to maintain a relationship among the respective characteristics for each of the multiple wavelength signals.

20. The system of claim 18, wherein the reference value corresponds to at least one of a carboxyhemoglobin value, a methemoglobin value, or an oxygenation value.

21-24. (canceled)
